(19) United States
(12) Patent Application Publication
(10) Pub. No.: US 2019/0392528 A1
(43) Pub. Date: Dec. 26, 2019

(54) SYSTEM FOR GENERATING A PET HEALTHCARE PLAN

(71) Applicant: Will Novak, Vancouver, WA (US)

(72) Inventor: Will Novak, Vancouver, WA (US)

(21) Appl. No.: 16/561,124

(22) Filed: Sep. 5, 2019

Related U.S. Application Data
(63) Continuation of application No. 15/598,710, filed on May 18, 2017.

(51) Int. Cl. G06Q 40/08; G16H 15/00; G16H 50/30

(52) U.S. Cl. CPC G06Q 40/08; G16H 10/60; G16H 50/30; G16H 15/00

(57) ABSTRACT

A computer implemented system for determining and generating a patient healthcare plan may include: a computing platform having a processor, a memory in communication with the processor, and input logic stored in the memory, executable by the processor and configured to receive input from the user, wherein the input comprises data for populating a patient data record stored in the memory; identification logic stored in the memory, executable by the processor and configured to identify from a database an impact factor corresponding to the data populating the patient data record; calculation logic stored in the memory, executable by the processor and configured to calculate a healthcare cost for a time period based on the identified impact factor; and generating logic stored in the memory, executable by the processor and configured to generate a patient healthcare plan having a healthcare item and having a number of healthcare credits that are accrued over the time period.

### CROSS REFERENCE TO RELATED APPLICATIONS

This application is a continuation of and claims priority to and the benefit of the filing date of U.S. Non-Provisional application Ser. No. 15/598,710, filed on May 18, 2017 which is hereby incorporated by reference in its entirety.

### FIELD OF THE INVENTION

This patent specification relates to the field of providing and generating healthcare information. More specifically, this patent specification relates to systems and methods that are configured to provide patient health care plans including plan information, costs, and usage, for pets and other living organisms.

### BACKGROUND

Currently, all pet health care plans are either a wellness program that does not cover accident or illness care or pet insurance that is regulated by the Department of Insurance (DOI) in each state. Pet insurance can cover accident and illness as well as wellness and typically includes deductibles, copays, and financial limitations on payments as well as a complex reimbursement process. Pet insurance also has a waiting period and will exclude pre-existing conditions. Wellness programs do not cover accident and illness services, but instead cover routine wellness veterinary visits and other non-medical services.

All too often, pet owners do not consider pet insurance or pet wellness programs as a good value for various reasons. For example, pet owners may be confused by not really knowing what service will be paid for when an event happens or by the complex reimbursement and payment calculation process. Pet care can be quite expensive to many pet owners and without the financial help of pet insurance and/or pet wellness programs, the owners may choose to forgo or limit immediate or preventative care for their pet. For this reason, both the owners and their pets may unnecessarily experience stress, discomfort, and medical complications that could otherwise be avoided.

Therefore, a need exists for novel computer-implemented systems and methods that are configured to provide information which includes patient health care plan information, costs, and usage. A further need exists for novel computer-implemented systems and methods for providing and generating patient healthcare information which covers accident and illness services, in addition to routine wellness veterinary visits and other non-medical services. Finally, a need exists for novel computer-implemented systems and methods for providing and generating patient healthcare information which provide the information and care reimbursement in an easily understood format.

### BRIEF SUMMARY OF THE INVENTION

A system and methods for determining and generating a patient healthcare plan are provided which is not classified an insurance product. Owners of pet patients may be billed for a selected healthcare plan via automated billing system and tracking preferably on a monthly or annual basis. Health care credits, exchangeable for healthcare items, may accrue each month and may roll over until used. The system may provide each owner with information describing the number many healthcare credits available for care and the wellness items used on the plan. The longer the pet remains healthy the more healthcare credits are available for when the pet has an illness.

In some embodiments, a computer implemented system for determining and generating a patient healthcare plan may include: a client device having a display screen and an input interface for receiving input from a user; a computing platform having a processor, a memory in communication with the processor, and input logic stored in the memory, executable by the processor and configured to receive input from the user, wherein the input comprises data for populating a patient data record stored in the memory; identification logic stored in the memory, executable by the processor and configured to identify from a database an impact factor corresponding to the data populating the patient data record; calculation logic stored in the memory, executable by the processor and configured to calculate a healthcare cost for a time period based on the identified impact factor; and generating logic stored in the memory, executable by the processor and configured to generate a patient healthcare plan having a healthcare item and having a number of healthcare credits that are accrued over the time period.

According to one embodiment consistent with the principles of the invention, a computer implemented method for determining and generating a patient healthcare plan may include the steps of: receiving input from a user through a client device, wherein the input comprises data for populating a patient data record; identifying from a database, via a computing device processor, an impact factor corresponding to the data populating the patient data record; calculating a healthcare cost for a time period based on the identified impact factor; and generating a patient healthcare plan having a healthcare item and having a number of healthcare credits that are accrued over the time period.

According to another embodiment consistent with the principles of the invention, a computer implemented method for generating a patient contract is provided and may include the steps of: receiving input for populating a patient data record; retrieving one or more healthcare plans; calculating a healthcare cost for a time period; receiving input indicating a selected healthcare plan; and generating a healthcare plan having a patient contract describing a healthcare item and a number of healthcare credits accrued over the time period.

According to another embodiment consistent with the principles of the invention, a computer implemented method for determining patient healthcare plan usage is provided and may include the steps of: receiving a service request having a healthcare item from a healthcare provider; determining if healthcare item matches a patient healthcare item; deducting a number of healthcare credits associated with the patient healthcare item from the patient healthcare credits; and providing the deducted number of healthcare credits to the healthcare provider.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts an illustrative example of some of the components and computer implemented methods which may be found in a system for determining and generating a patient healthcare plan according to various embodiments described herein.

FIG. 2 illustrates a block diagram showing an example of a server which may be used by the system as described in various embodiments herein.

FIG. 3 shows a block diagram illustrating an example of a client device which may be used by the system as described in various embodiments herein.

FIG. 4 depicts a block diagram illustrating some applications of a system for determining and generating a patient healthcare plan which may function as software rules engines according to various embodiments described herein.

FIG. 5 illustrates a block diagram illustrating an example of a system database according to various embodiments described herein.

FIG. 6 shows a block diagram illustrating an example of a patient data record according to various embodiments described herein.

FIG. 7 depicts a block diagram illustrating an example of a provider data record according to various embodiments described herein.

FIG. 8 illustrates a block diagram illustrating an example of an owner data record according to various embodiments described herein.

FIG. 9 shows a block diagram of an example of a computer-implemented method for generating a patient healthcare plan according to various embodiments described herein.

FIG. 10 depicts a block diagram of an example of a computer-implemented method for generating a patient contract according to various embodiments described herein.

FIG. 11 illustrates a block diagram illustrating an example of a computer-implemented method for determining patient healthcare plan usage according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

### Definitions

As used herein, the term “computer” refers to a machine, apparatus, or device that is capable of accepting and performing logic operations from software code. The term “application”, “software”, “software code” or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, or a voice-over-IP (VoIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New computer-implemented systems and methods for providing patient health care plans including plan information, costs, and usage are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

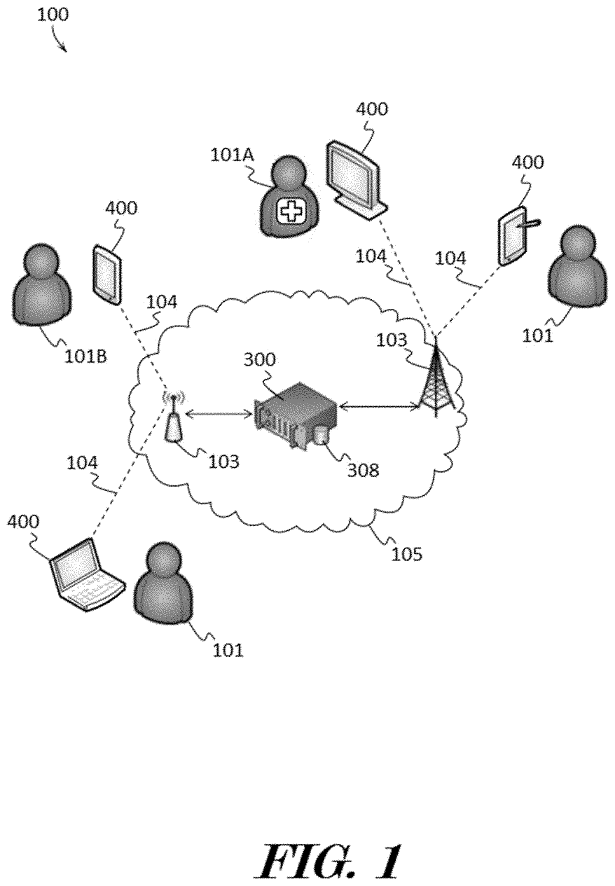

The present invention will now be described by example and through referencing the appended figures representing preferred and alternative embodiments. As perhaps best shown by FIG. 1, an illustrative example of some of the physical components which may comprise a system for determining and generating a patient healthcare plan (“the system”) 100 according to some embodiments is presented. In preferred embodiments, a patient may comprise a pet patient or animal patient, however, in other embodiments, a patient may comprise a human or any other living organism including plants. The system 100 is configured to facilitate the transfer of data and information between one or more access points 103, client devices 400, and servers 300 over a data network 105. Each client device 400 may send data to and receive data from the data network 105 through a network connection 104 with an access point 103. A data store 308 accessible by the server 300 may contain one or more databases. The data may comprise any information pertinent to one or more users 101 input into the system 100 including information on or describing one or more users 101, information on or describing one or more pets or animal patients, information on or describing one or more owners 101B of a pet or animal patient, information on or describing one or more healthcare providers 101A, information on or describing healthcare plans, information requested by one or more users 101, information supplied by one or more users 101, and any other information which a user 101 may be used to provide pet health care plans including plan information, costs, and usage.

In this example, the system 100 comprises at least one client device 400 (but preferably more than two client devices 400) configured to be operated by one or more users 101. Client devices 400 can be mobile devices, such as laptops, tablet computers, personal digital assistants, smart phones, and the like, that are equipped with a wireless network interface capable of sending data to one or more servers 300 with access to one or more data stores 308 over a network 105 such as a wireless local area network (WLAN). Additionally, client devices 400 can be fixed devices, such as desktops, workstations, and the like, that are equipped with a wireless or wired network interface capable of sending data to one or more servers 300 with access to one or more data stores 308 over a wireless or wired local area network 105. The present invention may be implemented on at least one client device 400 and/or server 300 programmed to perform one or more of the steps described herein. In some embodiments, more than one client device 400 and/or server 300 may be used, with each being programmed to carry out one or more steps of a method or process described herein.

In some embodiments, the system 100 may be configured to facilitate the communication of information to and from one or more users 101, through their respective client devices 400, and servers 300 of the system 100. Users 101 of the system 100 may include one or more healthcare providers 101A and owners 101B. A healthcare provider 101A may comprise an individual or entity that may provide healthcare and/or wellness services to a pet or animal patient, such as veterinarians and their support staff, animal hospitals, pet groomers and grooming entities, or any other individual or entity that may provide a service or tangible good to a pet or pet owner 101B. An owner 101B may comprise an individual or entity that may be responsible for, own, or otherwise have an interest in securing a services and/or tangible goods for a pet or animal patent, such as a dog, cat, ferret, hamster, rabbit, fish, parrot, turtle, snake, or any other animal that a human may keep as a pet or companion.

In preferred embodiments, the system 100 may be contain a database for pets, such as dogs and cats, that uses historical data of total pet health care costs which may be based on breed and age of pet, location of the healthcare provider 101A, healthcare provider 101A retail pricing, and consumer price index to determine the average cost of care for that pet in that general location. The system 100 may be configured to determine and generate one or more pet healthcare plans preferably at various plan levels that include a combination of healthcare items and healthcare credits that can be applied toward or exchanged for a healthcare item (service or tangible good) provided by a healthcare provider 101A. In further embodiments, the system 100 may calculate the amount of healthcare items provided within a healthcare plan as well as the additional healthcare credits that the specific age and breed of pet would need to cover higher levels of care for the general location of the healthcare provider 101A and retail pricing. In still further embodiments, the system 100 may track the usage of the healthcare items and the healthcare credits used for a healthcare plan for a pet. Healthcare credits may accrue over successive time periods, such as a month, and may roll over until used. In further preferred embodiments, the system 100 may provide data to the owner 101B of a pet which includes the number of healthcare credits available on the plan that the owner 101B has enrolled their pet into.

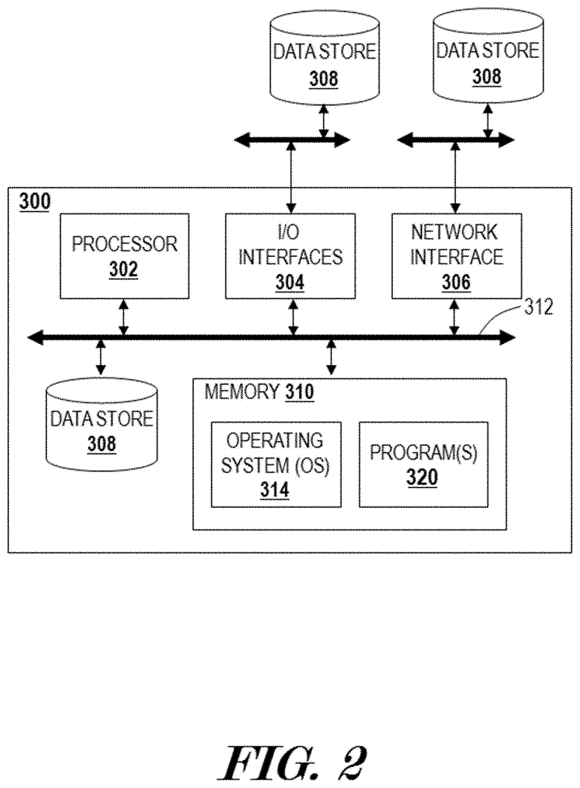

Referring now to FIG. 2, in an exemplary embodiment, a block diagram illustrates a server 300 of which one or more may be used in the system 100 or standalone and which may be a type of computing platform. The server 300 may be a digital computer that, in terms of hardware architecture, generally includes a processor 302, input/output (I/O) interfaces 304, a network interface 306, a data store 308, and memory 310. It should be appreciated by those of ordinary skill in the art that FIG. 2 depicts the server 300 in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 may be, for example but not limited to, one or more buses or other wired or wireless connections, as is known in the art. The local interface 312 may have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 may be any custom made or commercially available processor, a central processing unit (CPU), an auxiliary processor among several processors associated with the server 300, a semiconductor-based microprocessor (in the form of a microchip or chip set), or generally any device for executing software instructions. When the server 300 is in operation, the processor 302 is configured to execute software stored within the memory 310, to communicate data to and from the memory 310, and to generally control operations of the server 300 pursuant to the software instructions. The I/O interfaces 304 may be used to receive user input from and/or for providing system output to one or more devices or components. User input may be provided via, for example, a keyboard, touch pad, and/or a mouse. System output may be provided via a display device and a printer (not shown). I/O interfaces 304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 306 may be used to enable the server 300 to communicate on a network, such as the Internet, the data network 105, the enterprise, and the like, etc. The network interface 306 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 306 may include address, control, and/or data connections to enable appropriate communications on the network. A data store 308 may be used to store data.

The data store 308 is a type of memory and may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media. In one example, the data store 308 may be located internal to the server 300 such as, for example, an internal hard drive connected to the local interface 312 in the server 300. Additionally in another embodiment, the data store 308 may be located external to the server 300 such as, for example, an external hard drive connected to the I/O interfaces 304 (e.g., SCSI or USB connection). In a further embodiment, the data store 308 may be connected to the server 300 through a network, such as, for example, a network attached file server.

The memory 310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 may include one or more software programs, each of which includes an ordered listing of executable instructions for implementing logical functions. The software in the memory 310 may include a suitable operating system (O/S) 314 and one or more programs 320.

The operating system 314 essentially controls the execution of other computer programs, such as the one or more programs 320, and provides scheduling, input-output control, file and data management, memory management, and communication control and related services. The operating system 314 may be, for example Windows NT, Windows 2000, Windows XP, Windows Vista, Windows 7, Windows 8, Windows 10, Windows Server 2003/2008 (all available from Microsoft, Corp. of Redmond, Wash.), Solaris (available from Sun Microsystems, Inc. of Palo Alto, Calif.), LINUX (or another UNIX variant) (available from Red Hat of Raleigh, N.C. and various other vendors), Android and variants thereof (available from Google, Inc. of Mountain View, Calif.), Apple OS X and variants thereof (available from Apple, Inc. of Cupertino, Calif.), or the like. The one or more programs 320, may optionally include a communication application 111, a coordination engine 112, a calculation engine 113, and a plan management engine 114, and may be configured to implement the various processes, algorithms, methods, techniques, etc. described herein.

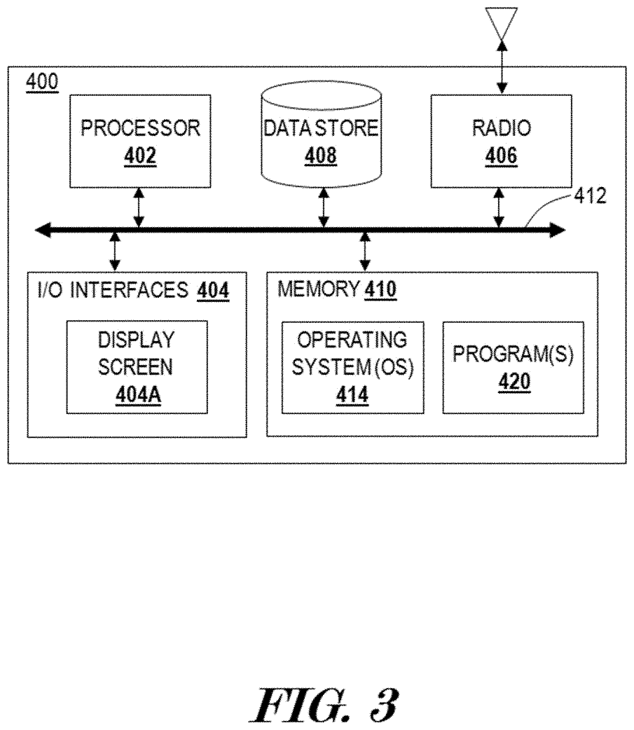

Referring to FIG. 3, in an exemplary embodiment, a block diagram illustrates a client device 400 of which one or more may be used in the system 100 or the like and which may be a type of computing platform. The client device 400 can be a digital device that, in terms of hardware architecture, generally includes a processor 402, input/output (I/O) interfaces 404, a radio 406, a data store 408, and memory 410. It should be appreciated by those of ordinary skill in the art that FIG. 3 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

The I/O interfaces 404 can be used to receive data and user input and/or for providing system output. User input can be provided via a plurality of I/O interfaces 404, such as a keypad, a touch screen, a camera, a microphone, a scroll ball, a scroll bar, buttons, bar code scanner, voice recognition, eye gesture, and the like. System output can be provided via a display screen 404A such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker or other sound emitting device configured to emit audio notifications, a vibrational device configured to vibrate, shake, or produce any other series of rapid and repeated movements to produce haptic notifications, and/or a light emitting diode (LED) or other light emitting element which may be configured to illuminate to provide a visual notification.

The radio 406 enables wireless communication to an external access device or network. Any number of suitable wireless data communication protocols, techniques, or methodologies can be supported by the radio 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions. In the example of FIG. 3, the software in the memory system 410 includes a suitable operating system (O/S) 414 and programs 420.

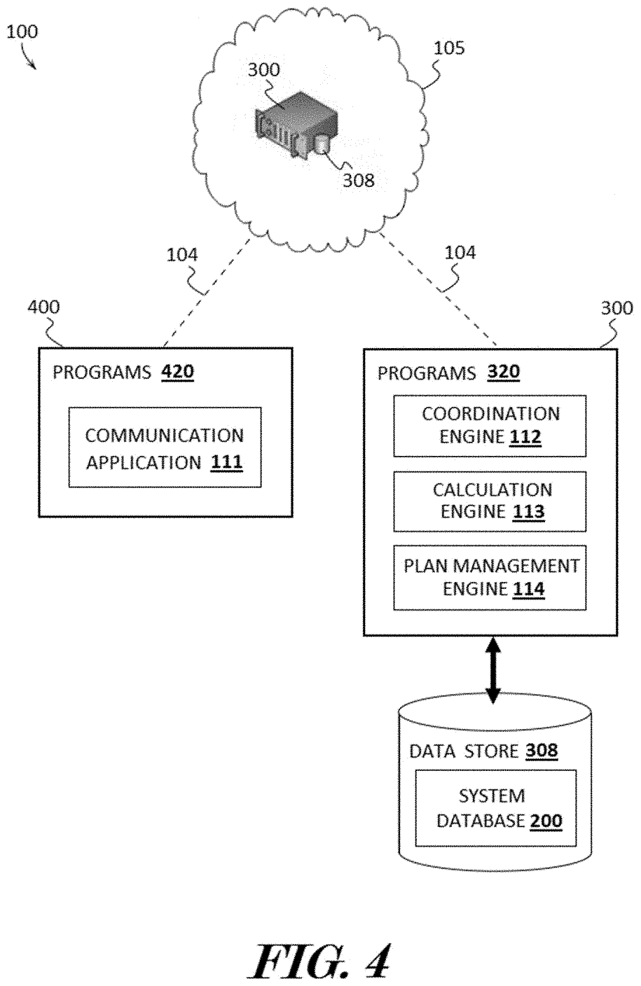

The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, Microsoft Windows 10, iOS (available from Apple, Inc.), webOS (available from Hewlett Packard), Blackberry OS (Available from Research in Motion), and the like. The programs 420 may include a communication application 421 (FIG. 4) and various applications, add-ons, etc. configured to provide end user functionality with the client device 400. For example, exemplary programs 420 may include, but not limited to, a web browser, social networking applications, streaming media applications, games, mapping and location applications, electronic mail applications, financial applications, and the like. The one or more programs 420, may optionally include a communication application 111, a coordination engine 112, a calculation engine 113, and a plan management engine 114, and may be configured to implement the various processes, algorithms, methods, techniques, etc. described herein. In a typical example, the end user typically uses one or more of the programs 420 along with a network 105 to manipulate information of the system 100.

Referring now to FIG. 4, a block diagram showing some software rules engines which may be found in a system 100 (FIG. 1), which may optionally be configured to run on a server 300 (FIGS. 1 and 2) and/or a client device 400 (FIGS. 1 and 3), and which may be in communication with a system database 200 according to various embodiments described herein are illustrated. In this and some embodiments, one or more servers 300 may be configured to run one or more software rules engines or programs such as a coordination engine 112, a calculation engine 113, and a plan management engine 114, while a client device 400 may be configured to run one or more software rules engines or programs such as a communication application 111. The server 300 and client device 400 may be in wired and/or wireless electronic communication, optionally through a network 105 via a network connection 104, with a data store 308 comprising a database, such as a system database 200. The engines 111, 112, 113, 114, may read, write, or otherwise access data in one or more databases of the data store 308. Additionally, the engines 111, 112, 113, 114, may be in electronic communication so that data may be readily exchanged between the engines 111, 112, 113, 114.

In other embodiments, a communication application 111, a coordination engine 112, a calculation engine 113, and a plan management engine 114, may be configured to run on one or more client devices 400 and/or servers 300 with data transferred to and from one or more servers 300 in communication with a data store 308 through a network 105. In still further embodiments, a server 300 or a client device 400 may be configured to run a communication application 111, a coordination engine 112, a calculation engine 113, and a plan management engine 114. It should be understood that the functions attributed to the engines 111, 112, 113, 114, described herein are exemplary in nature, and that in alternative embodiments, any function attributed to any engine 111, 112, 113, 114, may be performed by one or more other engines 111, 112, 113, 114.

Turning now to FIGS. 4-8, in some embodiments, the communication application 111 may comprise or function as input logic stored in the memory 310, 410, which may be executable by the processor 302, 402, of a server 300 and/or client device 400. The communication application 111 may receive input from a user 101, such as a healthcare provider 101A or an owner 101B, preferably through an I/O interface 304, 404, and the input may comprise information for populating a patient data record 202 of a patient database 201, information for populating a provider data record 222 of a provider database 221, and information for populating an owner data record 252 of an owner database 251. Optionally, the communication application 111 may read, write, edit, create, import, export, and delete information in a database of the system 100, such as the system database 200, patient database 201, provider database 221, healthcare database 231, and owner database 251.

In some embodiments, the coordination engine 112 may comprise or function as identification logic stored in the memory 310, 410, which may be executable by the processor 302, 402, of a server 300 and/or client device 400. The coordination engine 112 may manage queries of the system database 200, including any other databases 201, 221, 231, 251, in addition to being configured to search, identify, read, write, edit, create, import, export, and delete information in a database of the system 100, such as the system database 200, patient database 201, provider database 221, healthcare database 231, and owner database 251. For example, the coordination engine 112 may identify an impact factor 242, such as a geo-location factor 243 that describes the relative cost of healthcare items in central Florida, from the healthcare database 231 corresponding to data describing the home address of a pet as being in central Florida, from a patient data record 202.

In some embodiments, the calculation engine 113 may comprise or function as calculation logic stored in the memory 310, 410, which may be executable by the processor 302, 402, of a server 300 and/or client device 400. The calculation engine 113 may perform calculations and data manipulations which may be used to provide healthcare plan information and the like that may be outputted by the system 100. For example, calculation engine 113 may calculate a healthcare cost for a time period of a pet located in central Florida based on a geo-location factor 243 for pets of a similar breed and age to the pet located in central Florida.

In some embodiments, the plan management engine 114 may comprise or function as generating logic stored in the memory 310, 410, which may be executable by the processor 302, 402, of a server 300 and/or client device 400. The plan management engine 114 generate the healthcare plans provided by the system 100, with each health plan having one or more healthcare items and healthcare credits that are accrued over a time period. For example, plan management engine 114 may use the data provided by the coordination engine 112 and calculation engine 113 to generate a first healthcare plan, second healthcare plan, third healthcare plan, and fourth healthcare plan in which each healthcare plan has a successively greater number of healthcare items available to a pet and a greater number of healthcare credits are accrued over a time period. A time period, such as when used in a base cost for time period 236, a healthcare cost for a time period 215, or as otherwise used herein may describe a length or period of time, such as a day, a week, two weeks, three weeks, four weeks, a month, two months, three months, four months, five months, six months, a year, two years, three years, or any other length of time.

The system 100 may comprise one or more databases, such as a system database 200, optionally stored on a data store 308, 408, of one or more servers 300 and/or client device 400 accessible to a rules engine 321, instructional engine 322, and/or communication application 421. A system database 200 may comprise any data and information input to and output by the system 100. Data of the system database 200 may be stored or included one or more other databases such as a patient database 201, a provider database 221, and a healthcare database 231. It should be understood that the described structure of the system database 200 is exemplary in nature, and that in alternative embodiments, the data contained within the system database 200 may be organized in any other way.

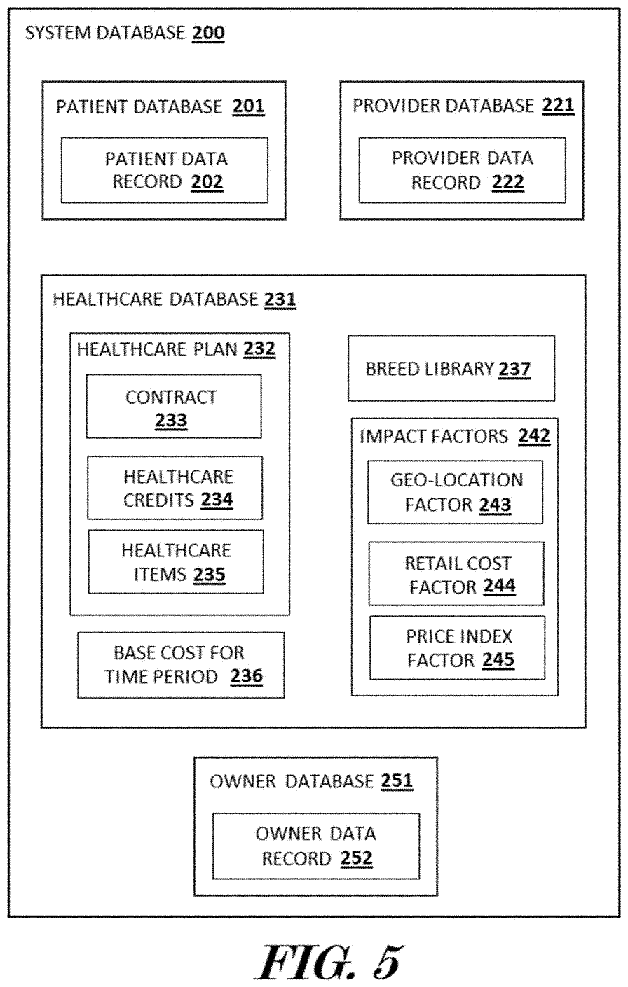

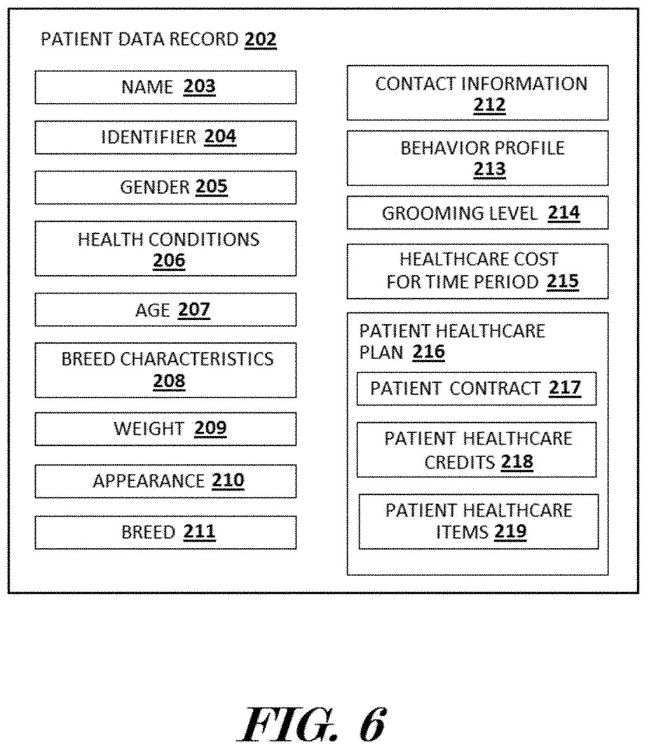

Turning now both FIGS. 5 and 6, the patient database 201 may comprise one or more patient data records 202 and each patient data record 202 may contain information in one or more data fields which may describe one or more parameters of a pet patient and a healthcare plan that the pet patient is enrolled in. These data fields may include data describing the pet patient that may include: the name 203; identifier 204; the gender 205; health conditions 206; age 207; breed characteristics 208; weight 209; appearance 210; breed 211; contact information 212; behavior profile 213; grooming level 214; healthcare cost for a time period 215; patient healthcare plan 216; patient contract 217; patient healthcare credits 218; patient healthcare items 219; and/or any other data fields or information.

The name 203 data field may contain information or data input by a user 101 which may describe the name of a pet patient which may be enrolled in the system 100 and able to receive healthcare items and healthcare credits.

The identifier 204 data field may contain information or data input by a user 101 which may describe an alphanumeric string or ID number which may be unique to the pet.

The gender 205 data field may contain information or data input by a user 101 which may describe the sex of the pet and/or information describing if the pet is spayed or neutered.

The health conditions 206 data field may contain information or data input by a user 101 which may describe any health conditions, diseases, or other information descriptive of the physical condition of the pet.

The age 207 data field may contain information or data input by a user 101 which may describe the current age of the pet. Additionally, the age 207 data field may contain the date of birth or other similar dating information for the pet.

The breed characteristics 208 data field may contain information or data input by the coordination engine 112, as retrieved from the breed library 237, which may describe any characteristic of the breed identified in breed 211, such as temperamental characteristics, physical characteristics, needs, suggestions, or other information which may be particular to the breed of the pet.

The weight 209 data field may contain information or data input by a user 101 which may describe the weight or approximate weight of the pet, including weight history.

The appearance 210 data field may contain information or data input by a user 101 which may describe the physical appearance of the pet.

The breed 211 data field may contain information or data input by a user 101 which may describe the pet as defined by breed standards for the industry.

The contact information 212 data field may contain information or data input by a user 101 which may describe a home address, billing address, or other address of the pet. Optionally, the contact information 212 data field may contain information linking the pet of the pet data record to an owner data record 252.

The behavior profile 213 data field may contain information or data input by a user 101 which may describe behavior traits, behavior characteristics, and/or temperament information which may be particular to the pet.

The grooming level 214 data field may contain information or data input by the coordination engine 112, as retrieved from the breed library 237, which may describe a type or amount of grooming which the pet may receive.

The healthcare cost for a time period 215 data field may contain information or data output by the calculation engine 113 which may include a monetary amount or value corresponding to the average cost of care for that pet in that general location. The calculation engine 113 may use historical data of total pet health care costs which may be based on breed and age of pet, location of a healthcare provider 101A, healthcare provider 101A retail pricing, and consumer price index to determine the average cost of care for that pet in that general location.

The patient healthcare plan 216 data field may contain information or data output by the plan management engine 114 which may include a data describing a healthcare plan that a pet is or may be enrolled in such as enrollment date, termination date, expiration date, name of the healthcare plan, options for the healthcare plan, or any other information. Additionally, this data may include a patient contract 217, a patient healthcare credits data field 218, and a patient healthcare items 219 data field. The patient contract 217 may comprise a copy of a contract entered into by the owner 101B of the pet patient describing the offer, acceptance, consideration, mutuality of obligation, competency and capacity, and, optionally, a written instrument, entered into by an owner 101B and the entity administrating the healthcare plan. The patient healthcare credits data field 218 may contain information or data describing the number of healthcare credits earned, exchanged, or to be accumulated in a time period. The patient healthcare items 219 data field may contain information or data describing the healthcare items available, redeemed, or to be accumulated in a time period.

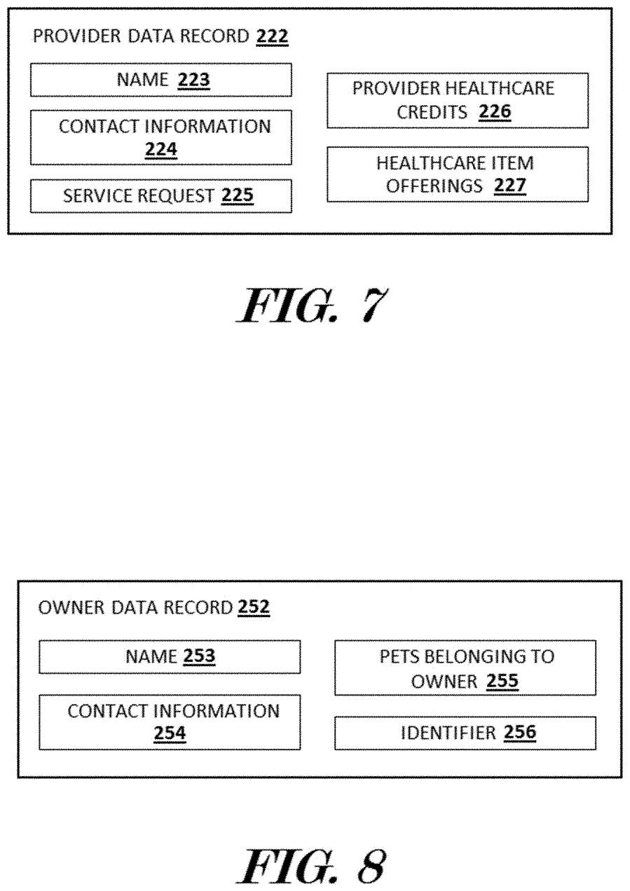

Turning now to FIGS. 5 and 7, the provider database 221 may comprise one or more provider data records 222 and each provider data record 222 may contain information in one or more data fields which may describe one or more parameters of a healthcare provider 101A. A healthcare provider 101A may refer generally to any individual or entity that is able to provide healthcare items to a pet or to the owner 101A of the pet. These data fields may include: name 223; contact information 224; service requests 225; provider healthcare credits 226; healthcare item offerings 227; and/or any other data fields or information.

The name 223 data field may contain information or data input by a user 101 which may describe the name of a healthcare provider 101A individual or entity which may be enrolled in the system 100 and able to provide healthcare items and receive healthcare credits.

The contact information 224 data field may contain information or data input by a user 101 which may describe a business address, billing address, or other address of the healthcare provider 101A. Additionally, the contact information 224 data field may contain phone numbers, fax numbers, email addresses, or other messaging information which may be used to contact a healthcare provider 101A.

The service requests 225 data field may contain information or data input by a user 101 which may describe one or more healthcare items that were or are to be provided to a pet patient or their owner 101B and which the healthcare provider 101A is requesting reimbursement for, preferably reimbursement in the form of healthcare credits.

The provider healthcare credits 226 data field may contain information or data which may describe one or more healthcare credits that have been or are to be provided to a healthcare provider 101A in exchange for providing healthcare items to a pet patient or their owner 101B.

The healthcare item offerings 227 data field may contain information or data which may describe one or more healthcare items which the healthcare provider 101A may be capable of providing to a pet patient or their owner 101B. Healthcare items may include health exams, grooming services, medication, supplements, or any other service or tangible good that may be provided to a pet or pet owner 101B.

As shown in FIG. 5, the system database 200 may include a healthcare database 231 which may include data and information in one or more data fields which may be used to provide pet health care plans including plan information, costs, and usage and which may contain information may describe one or more parameters of a healthcare provider 101A. These data fields may include: healthcare plan 232; contract 233; healthcare credits 234; healthcare items 235; base cost for time period 236; breed library 237; impact factors 242; geo-location factor 243; retail cost factor 244; price index factor 245; and/or any other data fields or information.

The healthcare plan 232 data field may contain information or data input by a user 101 which may describe the name and other details of a healthcare plan. The healthcare plan 232 may include a contract 233, one or more healthcare credits 234 earned or accumulated over a time period, and one or more healthcare items 235 which may be used in a time period and/or exchanged for one or more of the healthcare credits 234 earned or accumulated over a time period. The contract 233 of a healthcare plan 232 may be generic and once populated with data specific to a pet and their owner 101B, the healthcare plan 232 may be saved in the patient data record 202 by an engine, such as the plan management engine 114. The healthcare database 231 may comprise any number of healthcare plans 232 with each healthcare plan 232 having a different contract 233, different number of healthcare credits 234 accumulated in a time period, and/or different number of or different available healthcare items 235.

The base cost for time period 236 data field may contain information or data input by a user 101 and/or data output by the calculation engine 113 which may describe a monetary amount or value corresponding to the average cost of care for a breed and age of a pet. In preferred embodiments, the healthcare database 231 may comprise a plurality of base cost for time period 236 data fields that may each describe a pet having a breed or mix of breeds for one or more time periods of the life of the pet. For example, a base cost for time period 236 may be twelve dollars per month for a poodle that is five to six years of age.

The breed library 237 data field may contain information or data which may describe characteristics and other information which may be descriptive of one or more, and preferably a plurality, of breeds of animals. In some embodiments, the breed library 237 may contain a grooming level associated with each breed of animal contained in the breed library 237. In further embodiments, the breed library 237 may contain one or more breed characteristics associated with each breed of animal contained in the breed library 237.

The impact factors 242 data field may contain information or data input by a user 101 and/or data output by the calculation engine 113 which may describe one or more factors which may influence or be used to calculate a healthcare cost for a time period 215 for a pet patient which may have data stored in the patient data record 202. One or more impact factors 242 may be mathematically combined with the base cost for time period 236 by the calculation engine 113 to generate a healthcare cost for a time period 215 for a pet. These impact factors 242 may be selected by the coordination engine 112 based on information contained in the patient data record 202. Impact factors 242 may include one or more geo-location factors 243, retail cost factors 244, price index factors 245, and/or any other factors which may be mathematically combined with the base cost for time period 236 to arrive at a healthcare cost for a time period 215 for a pet.

The geo-location factor 243 data field may contain information or data input by a user 101 and/or data output by the calculation engine 113 which may describe the average cost for healthcare items in an area or location relative to the average cost for healthcare items in a larger area. For example, the average cost for a healthcare item in Buffalo, N.Y. may be 5% lower than the average cost for the same or similar healthcare item in the United States resulting in a geo-location factor 243 of 0.95.

The retail cost factor 244 data field may contain information or data input by a user 101 and/or data output by the calculation engine 113 which may describe the average cost for healthcare items provided by one or more healthcare providers 101A relative to the average cost for healthcare items provided by healthcare providers 101A in a larger area. For example, the average cost for a healthcare item at a specific healthcare provider 101A animal hospital may be 50% higher than the average cost for the same or similar healthcare item provided by a plurality of healthcare providers 101A in the country of Spain resulting in a retail cost factor 244 of 1.50.

The price index factor 245 data field may contain information or data input by a user 101 and/or data output by the calculation engine 113 which may describe a measure of the consumer price index (CPI) which measures changes in the price level of market basket of consumer goods and services purchased by households. For example, the current consumer price index (CPI) may be 1% higher than the previous year's CPI (or the year in which the base cost for time period 236 was determined) resulting in a price index factor 245 of 1.01.

Turning now to FIGS. 5 and 8, the owner database 251 may comprise one or more owner data records 252 and each owner data record 252 may contain information in one or more data fields which may describe one or more parameters of an owner 101B. An owner 101B may refer generally to any individual or entity that is able to own a pet and pay for healthcare items for the pet. These data fields may include: name 253; contact information 254; pets belonging to owner 255; and/or any other data fields or information.

The name 253 data field may contain information or data input by a user 101 which may describe the name of an owner 101A which may be an individual or entity which own one or more pets that may be enrolled in the system 100.

The contact information 254 data field may contain information or data input by a user 101 which may describe a home address, billing address, or other address of the owner 101B. Additionally, the contact information 254 data field may contain phone numbers, fax numbers, email addresses, or other messaging information which may be used to contact an owner 101B.

The pets belonging to owner 255 data field may contain information or data which may describe and identify the one or more pets that may be enrolled in the system 100 that belong to the owner 101B.

The identifier 256 data field may contain information or data input by a user 101 which may describe an alphanumeric string or ID number which may be unique to the owner 101B of a pet.

In preferred embodiments, the patient data records 202 may be separate from the owner data records 252. One or more patient data records 202 may be associated (such as by associating the patient identifiers 204 with the owner identifier 256, with an owner data record 252 of the describing the owner 101B of the pet(s) owned by that pet owner 101B. This allows a pet to be transfer from one pet owner 101B to another. This also allows a pet owner 101B to have more than one pet associated their owner data record 252. The pet patient can have only one plan at a time but the owner 101B can have multiple pet patients, which each having a separate patient data record 202. This also facilitates the ability to change a pet patient to a different healthcare plan.

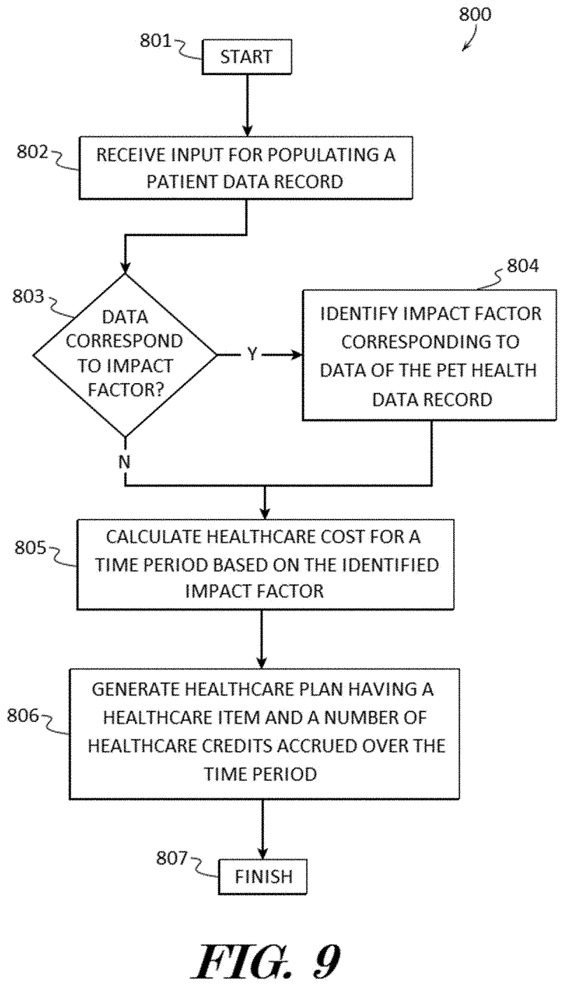

A block diagram of an example of a method for generating a patient healthcare plan (“the method”) 800 according to various embodiments described herein is shown as an overview in FIG. 9. The method 800 may be accomplished with one or more client devices 400 and/or servers 300. In some embodiments, the method 800 may start 801 and input for populating a patient data record 202 may be received in step 802. A patient data record 202 may be created for each pet patient and/or for each owner 101B of one or more pets in a patient database 201. In further embodiments, data populating one or more fields of the patient data record 202 may be input by a user 101 through a communication application 111 preferably running on a client device 400. In further embodiments, this data may include: the name 203; identifier 204; the gender 205; health conditions 206; age 207; breed characteristics 208; weight 209; appearance 210; breed 211; contact information 212; behavior profile 213; grooming level 214; healthcare cost for a time period 215; patient healthcare plan 216; patient contract 217; patient healthcare credits 218; patient healthcare items 219; and/or any other data fields or information. For example, data populating an age 207 data field may be five years and data populating a breed 211 data field may be beagle.

In decision block 803, the system 100 may determine if data populating the patient health data record 202 corresponds to an impact factor 242. In some embodiments, the coordination engine 112 may search the patient health data record 202 which may be stored in a patient database 201 and also search the impact factors 242, such as a geo-location factor 243, retail cost factor 244, and price index factor 245, stored in a healthcare database 231 to determine if data populating the patient health data record 202 corresponds to an impact factor 242. For example, the coordination engine 112 may search the patient health data record 202 for an address that is located in or associated with an area or location described by a geo-location factor 243. If the data populating the patient health data record 202 does not correspond to an impact factor 242, the method 800 may continue to step 805. If the data populating the patient health data record 202 does correspond to an impact factor 242, the method 800 may continue to step 804.

In step 804, an impact factor 242 corresponding to the data populating the patient data record 202 may be identified. In some embodiments, the coordination engine 112 may identify each impact factor 242 that corresponds to the data populating the patient data record 202. For example, if the patient health data record 202 comprises an address located in central Ohio, the coordination engine 112 may identify a geo-location factor 243 of 1.1 describing that the average cost for healthcare items in central Ohio is approximately 10% greater than in the United States as a whole. The identified impact factors 242 may be provided or otherwise made available to the calculation engine 113.

In step 805, a healthcare cost for a time period 215 may be calculated preferably based on the one or more impact factors 242 identified in step 804. In some embodiments, the calculation engine 113 may mathematically combine the one or more impact factors 242 with a base cost for time period 236 to calculate a healthcare cost for a time period 215. The base cost for time period 236 may be retrieved from the healthcare database 231 and may describe a base or average cost for a pet, preferably by age and one or more selected breed types. In preferred embodiments, two or more impact factors 242, each corresponding to the data populating the patient data record 202, may be identified by the coordination engine 112 from the system database 200, and each impact factor 242 may be additive to the healthcare cost for a time period 215 calculated by the calculation engine 113. Continuing the above example, for five year old beagles in central Ohio, the base cost for time period 236 may be fifteen dollars a month and the calculation engine 113 may mathematically combine a geo-location factor 243 of 1.1 with fifteen dollars a month to arrive at a healthcare cost for a time period 215 of $16.50 dollars a month for the five year old beagle to which the patient data record 202 describes.

In step 806, a patient healthcare plan 216 having one or more healthcare items 219 and having a number of patient healthcare credits 218 accrued over the time period may be generated. In some embodiments, the plan management engine 114 may generate the patient healthcare plan 216 and the communication application 111 may display the patient healthcare plan 216 to a user 101, via a display screen 404A of the client device 400 of the user 101. In further embodiments, the plan management engine 114 may generate the patient healthcare plan 216 by using data from a healthcare plan 232 which may include a contract 233, healthcare credits 234, and healthcare items 235. In still further embodiments, two or more patient healthcare plans 216 may be generated by the plan management engine 114, and each patient healthcare plan 216 may have a different number of patient healthcare credits 218 that are accrued over the same time period. The data from the patient data record 202, in addition to data calculated in step 805, may be used to populate and generate a patient healthcare plan 216 comprising a patient contract 217, patient healthcare credits 218, and patient healthcare items 219. For example, the breed 211, weight 209, and age 207 may be compared to base cost for time period 236 and a juvenile or adult plan type may be generated in which the weight 209 may determine optional medications (healthcare items 235) included in the generated plan. Continuing the above example, upon the receiving the $16.50 dollars a month, the plan management engine 114 may credit the patient healthcare plan 216 with 165 points every month which may be exchanged for one or more patient healthcare items 219. Once the patient healthcare plan 216 has been generated, the method 800 may finish 807.

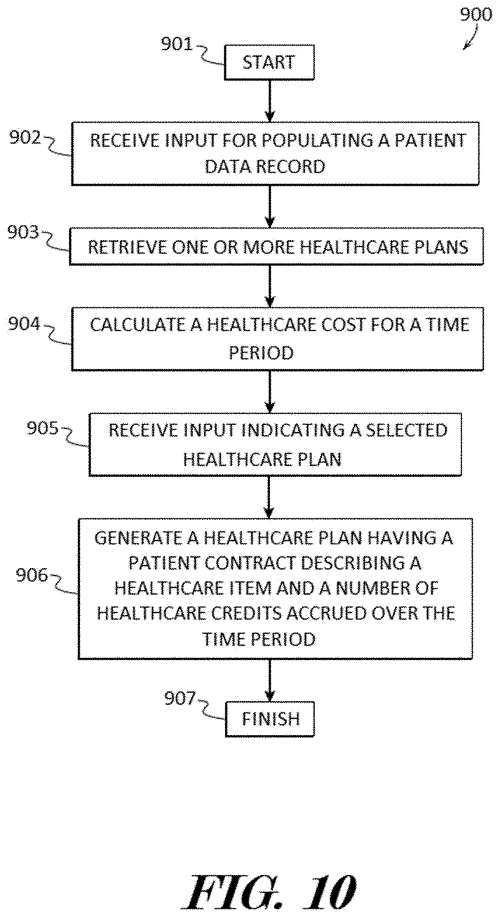

FIG. 10 depicts a block diagram of a method for generating a patient contract (“the method”) 900 according to various embodiments described herein. The method 900 may be accomplished with one or more client devices 400 and/or servers 300. In some embodiments, the method 900 may start 901 and input for populating a patient data record 202 may be received in step 902. A patient data record 202 may be created for each pet patient and/or for each owner 101B of one or more pets in a patient database 201. In further embodiments, data populating one or more fields of the patient data record 202 may be input by a user 101 through a communication application 111 preferably running on a client device 400. In further embodiments, this data may include: the name 203; identifier 204; the gender 205; health conditions 206; age 207; breed characteristics 208; weight 209; appearance 210; breed 211; contact information 212; behavior profile 213; grooming level 214; healthcare cost for a time period 215; patient healthcare plan 216; patient contract 217; patient healthcare credits 218; patient healthcare items 219; and/or any other data fields or information. For example, data populating an age 207 data field may be five years and data populating a breed 211 data field may be Persian cat.

In step 903, one or more healthcare plans 232 may be retrieved from the system database 200 by the coordination engine 112. In preferred embodiments, a healthcare database 231 of a system database 200 may comprise two or more healthcare plans 232 with each healthcare plan 232 having a contract 233, one or more healthcare credits 234, and one or more healthcare items 235 that are different from the other healthcare plans 232. For example, a first healthcare plan 232 may have lower costs, provide a lower number of healthcare credits 234 accrued over a time period, and a fewer number of healthcare items 235 than a second healthcare plan 232.

In step 904 a healthcare cost for a time period 215 may be calculated for each healthcare plan 232 retrieved in step 903. In some embodiments, the calculation engine 113 may mathematically combine one or more impact factors 242 with a base cost for time period 236 to calculate a healthcare cost for a time period 215. The base cost for time period 236 may be retrieved from the healthcare database 231 and may describe a base or average cost for a pet, preferably by age and one or more selected breed types. In preferred embodiments, two or more impact factors 242, each corresponding to the data populating the patient data record 202, may be identified by the coordination engine 112 from the system database 200, and each impact factor 242 may be additive or multiplied together with the base cost for time period 236 calculate to the healthcare cost for a time period 215.

In step 905, input indicating a healthcare plan 232 selected by a user 101, such as an owner 101B, may be received by the system 100. The input may be provided by a user 101 through a communication application 111 preferably running on a client device 400. Preferably, this input may comprise a digital signature or any a type of electronic signature that encrypts documents with digital codes that are particularly difficult to duplicate. In other embodiments, input indicating a healthcare plan 232 may comprise scans or digital copies of physical copies documents containing the signature of an owner 101B. In still further embodiments, the input indicating a healthcare plan 232 selected by a user 101 may include optional services, healthcare items 235 Additional HealthCare Credit accumulation rates, or other options which may be added onto or into a patient healthcare plan 216.

Next in step 906, a patient healthcare plan 216 having the patient contract 217 describing one or more patient healthcare items 219 and a number of patient healthcare credits 218 accrued over the time period may be generate by the plan management engine 114. The patient contract 217 may be generated for healthcare plan 232 selected by the user 101 in step 905. Once the patient healthcare plan 216 and patient contract 217 have been generated, the method 900 may finish 907.

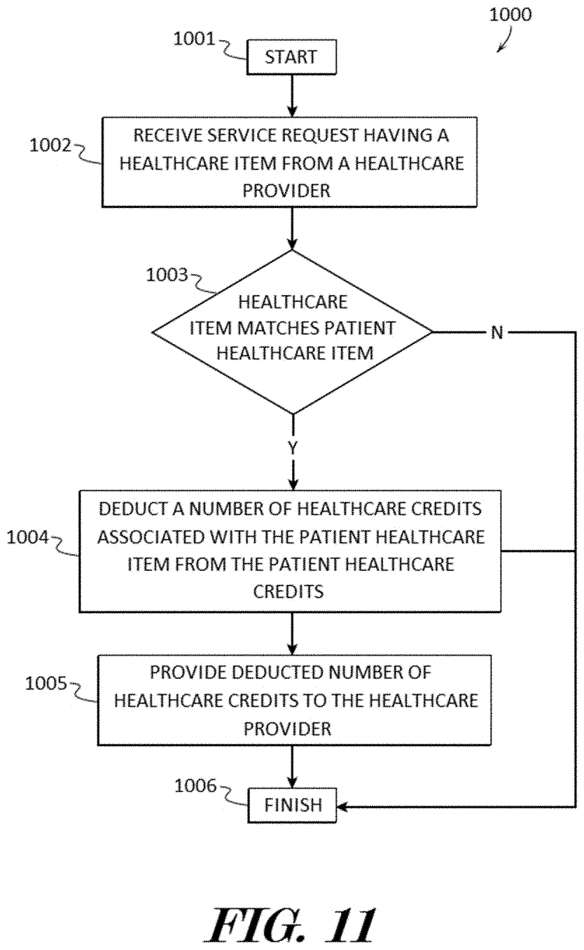

FIG. 11 illustrates a block diagram of a method for determining patient healthcare plan usage (“the method”) 1000 according to various embodiments described herein. The method 1000 may be accomplished with one or more client devices 400 and/or servers 300. In some embodiments, the method 1000 may start 1001 and a service request 225 may be received by the system 100 from a healthcare provider 101A in step 1002. The service request 225 may be provided by a user 101 through a communication application 111 preferably running on a client device 400. Generally, a service request 225 may include one or more healthcare items which may be provided to a pet patient or their owner 101B. The service request 225 may also include the name 223 and contact information 224 of the healthcare provider 101A and a name 203 and/or identifier 204 of a pet patient. For example, a service request 225 may include a request for the healthcare item of claw trimming service which the healthcare provider 101A desires or has performed on a pet patient.

In decision block 1003, the system 100 may determine if the healthcare item of the service request 225 matches a patient healthcare item 219 of the patient data record 202. In some embodiments, the coordination engine 112 may query the patient data record 202 identified by name 203 and/or identifier 204 of a pet patient in the service request 225 to see if the healthcare item of the service request 225 matches a patient healthcare item 219 of the patient data record 202. If the healthcare item of the service request 225 does not match the patient healthcare item 219 of the patient data record 202, the method 1000 may finish 1006. For example, if the patient data record 202 does not include the patient healthcare item 219 of claw trimming service, the method 1000 may finish 1006. If the healthcare item of the service request 225 does match the patient healthcare item 219 of the patient data record 202, the method 1000 may continue to step 1004.

In step 1004, the system 100 may deduct a number of healthcare credits associated with the patient healthcare item 219 from the patient healthcare credits 218. Generally, each patient healthcare item 219 may be exchangeable for one or more healthcare credits 218 so that the whole cost or a portion of the cost of a healthcare item in a service request 225 may be covered by the patient healthcare credits 218 that have accrued in the patient healthcare credits 218 of the patient data record 202. The calculation engine 113 and/or the plan management engine 114 may deduct the number of healthcare credits associated with the patient healthcare item 219 from the patient healthcare credits 218. Continuing the above example, if the pet patient has 500 patient healthcare credits 218, and the patient healthcare item 219 of claw trimming service is associated with 300 healthcare credits, the calculation engine 113 and/or the plan management engine 114 may deduct 300 healthcare credits from the 500 patient healthcare credits 218 so that the pet patient has 200 patient healthcare credits 218 associated with their patient data record 202. Preferably, the coordination engine 112 may update the number of patient healthcare credits 218 in the patient data record 202. In some embodiments, the system 100 may provide a monetary amount corresponding to the deducted healthcare credits to the healthcare provider 101A such as through a check, money transfer, or the like. Optionally, after step 1004, the method 1000 may finish 1006.

Optionally, the system 100 may provide the deducted number of healthcare credits to the healthcare provider 101A in step 1005. In some embodiments, the coordination engine 112 may credit or otherwise add the deducted number of healthcare credits to the provider healthcare credits 226 in the provider data record 222 of the healthcare provider 101A. In further embodiments, provider healthcare credits 226 in the provider data record 222 of the healthcare provider 101A may be exchanged or converted into cash or other forms of payment and reimbursed to the healthcare provider 101A. After step 1005, the method 1000 may finish 1006.

Turning now to Table 1, an example of a patient healthcare plan calculations is shown.

TABLE 1
Example Patient Healthcare Plan Calculations

| | Plan Level 1 | Plan Level 2 | Plan Level 3 | Plan Level 4 |
|---|---|---|---|---|
| $ Health Care Credits/Points included | $10/mo. or | $12/mo. or | $14/mo. or | $16/mo. or |
| | 100 points | 120 points | 140 points | 160 points |
| Buster's Health Care $ Retail Cost/mo. | $44.88 | $44.88 | $44.88 | $44.88 |
| based on comparison to database | | | | |
| % of Buster's Health Covered included in | 22% = | 27% = | 31% = | 36% = |
| Plan | $10/mo. | $12/mo. | $14/mo. | $16/mo. |
| | divided by | divided by | divided by | divided by |
| | $44.88/mo. | $44.88/mo. | $44.88/mo. | $44.88/mo. |
| Cost to Cover 70% of Buster's Health Care | ($44.88 − | ($44.88 − | ($44.88 − | ($44.88 − |
| for each plan level | $10) × 70% = | $12) × 70% = | $14) × 70% = | $16) × 70% = |
| | $24.42 | $23.02 | $21.62 | $20.22 |
| Free Care Added to Health Plan = 10% of | $24.42 × .1 = | $23.02 × .1 = $2.30 | $21.62 × .1 = | $20.22 × .1 = |
| 70% $ calculation | $2.44 | | $2.16 | $2.02 |
| Total $ of Health Care Credits for 70% | $24.42 + $2.44 = | $23.02 + $2.30 = | $21.62 + $2.16 = | $20.22 + $2.02 = |
| Protection Option | $26.86 | $25.32 | $23.78 | $22.24 |
| Total $ Health Care Credits for Plan level | $10 + $26.86 = | $25.32 + $12 = | $23.78 + $14 = | $22.24 + $16 = |
| with 70% Protection Option (HCC included | $36.86 | $37.32 | $37.78 | $38.24 |
| Plus 70%) | | | | |

In this example, the owner's 101B name may be Sally Smith and the name 203 of the pet patient may be Buster. Buster may be assigned a patient data record 202 having the name 203 of Buster, the age 207 of two years old, breed 211 of Labrador Retriever, and contact information 212 an owner 101B of Sally Smith and having a home address of with a city and state of Dallas, Tex.

Buster may be proximate to an animal hospital located in Dallas Tex. The geo-location for cost of health care is 10% higher in Dallas, Tex. than the average for the United States resulting in a geo-location factor 243 for the location of 1.1. The retail cost factor 244 for specific Hospital in Dallas, Tex. to provide treatment to Buster may be 2 as the retail price of health care is twice the cost of health care. Additionally, the Consumer Price Index may be 2% higher than prior year resulting in a price index factor 245 of 1.02. The Breed 211 and Age 207 of Buster may be compared to the base cost for time period 236 in the healthcare database 231 to retrieve an average cost to provide health care per month for ANY Labrador Retriever that is 2 yrs of Age is $20 (Average for the US based on Year 2016).

The system 100 may then calculate monthly health care cost at retail pricing for Labrador retriever, for hospital location in Dallas Tex. based on the impact factors 242 as:

$20 per month (D)×1.1 (A)×2 (B)×1.02 (C)=$44.88 per month.

Plan Level 1 may contain two types of healthcare items 235, prepackaged services plus basic health care protection for a cost of $10 per month resulting in 100 healthcare credits accrued over the time period of a month.

Plan Level 2 may contain two types of healthcare items 235, prepackaged services plus basic health care protection for a cost of $12 per month resulting in 120 healthcare credits accrued over the time period of a month.

Plan Level 3 may contain two types of healthcare items 235, prepackaged services plus basic health care protection for a cost of $14 per month resulting in 140 healthcare credits accrued over the time period of a month.

Plan Level 4 may contain two types of healthcare items 235, prepackaged services plus basic health care protection for a cost of $16 per month resulting in 160 healthcare credits accrued over the time period of a month.

The owner 101B may selects one of the Plan Levels from 1, 2, 3, 4, and one of a protection level from Basic, 70%, 80%, 90%. In some embodiments, the system 100 may select Adult or Juvenile plan type based on age of pet being less than or greater than 365 days of age.

In this example, owner 101B selects Level 2 and 70% protection level. The system 100 determines pet is greater than 365 days of age so Adult Plan Type selected.

The dollar amount of Health Care Credits per time period of a month in Adult Type, Plan Level 2 with 70% Protection will be $37.32. Therefore, $37.32 per month may be accrued in the pet health care account. Preferably, the dollar amount may be converted to a point system, such as one dollar equals ten points. However, healthcare credits, such as patient healthcare credits 218, healthcare credits 234, and provider healthcare credits 226 may be stored in the system database 200 and dollars, other currencies, points, or any other countable entity.

Continuing the example, after a 10 months duration of the plan for Buster, Labrador Retriever that is 2 yrs. of age=$37.72×10 month for total of $377.20 patient healthcare credits 218 available. Buster may contract a pet health illness, such as an ear infection. A healthcare provider 101A may order healthcare items to treat ear infection in a service request 225. These healthcare items may include Pet examination fee with a cost of $30, Ear Medication with a cost of $40, and Flea Medication with a cost of $20.

The system 100 may check the patient healthcare plan 216 selected by Buster's owner 101B for patient healthcare items 219 that patient healthcare credits 218 can be used for in Buster's patient healthcare plan 216 as agreed to in the patient contract 217.

In this example, Pet Examination is an included patient healthcare item 219, Ear Medications is an included patient healthcare item 219, and Flea Medication is an EXCLUDED patient healthcare item 219 or patient healthcare item 219 not in Buster's patient healthcare plan 216. The system 100 may credit the service requests 225 invoice for included patient healthcare items 219 only. The patient healthcare credits 218 available is $377.20 with the Pet Exam Included Item costs $30 and the Ear Medication Included Item costs $40.

The system 100 may calculate $377.20 (Available Health Care Credits) minus $30 minus $40=$307.20 of patient healthcare credits 218 remaining in the patient healthcare plan 216 so that the patient healthcare credits 218 available for Buster's next illness=$307.20. After six one month time periods have elapsed, $226.32 patient healthcare credits 218 may be accrued with a new total for available patient healthcare credits 218 calculated by $307.20+($37.72×6 months)=$533.52.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g. through a wireless cellular network or wifi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A system for determining and generating a patient healthcare plan based on at least a breed and an age of the patient, the system comprising:
a client device having a display screen and an input interface for receiving input from a user;
a computing platform having a processor, a memory in communication with the processor, and
input logic stored in the memory, executable by the processor and configured to receive input from the user, wherein the input comprises data for populating a patient data record stored in the memory;
identification logic stored in the memory, executable by the processor and configured to identify from a database an impact factor corresponding to the data populating the patient data record;
calculation logic stored in the memory, executable by the processor and configured to calculate a healthcare cost for a time period based on the identified impact factor; and
generating logic stored in the memory, executable by the processor and configured to generate a patient healthcare plan having a healthcare item and having a number of healthcare credits that are accrued over the time period.

2. The system of claim 1, wherein the impact factor is selected from the group consisting of a geo-location factor, a retail cost factor, and a consumer price index factor.

3. The system of claim 1, wherein the healthcare credits are exchangeable for the healthcare item.

4. The system of claim 1, wherein two or more impact factors corresponding to the data populating the patient data record are identified from the database, and wherein each impact factor is additive to the calculated healthcare cost for a time period.

5. The system of claim 1, wherein the generating logic is configured to generate two or more patient healthcare plans, and wherein each healthcare plan has a different number of healthcare credits that are accrued over the time period.

6. The system of claim 5, wherein the input logic is further configured to receive input from a user through a client device, in which the input identifies a healthcare plan selected by the user; and wherein the generating logic is configured to generate a contract for the selected healthcare plan.

7. The system of claim 1, wherein the time period is selected from the group consisting of: a week, two weeks, three weeks, four weeks, a month, two months, three months, four months, five months, six months, and a year.

8. The system of claim 1, wherein calculation logic is configured to calculate the healthcare cost for a time period by retrieving a base cost per time period, the base cost per time period associated with the data populating the patient data record, and wherein the base cost per time period and identified impact factor are multiplied together by the calculation logic to calculate the healthcare cost for a time period.

9. The system of claim 1, wherein the input logic is further configured to receive a service request for the healthcare item; and wherein the generating logic is further configured to deduct a number of healthcare credits associated in the database with the healthcare item from the accrued healthcare credits.

10. The system of claim 9, wherein the service request for the healthcare item is received from a healthcare provider, and wherein the generating logic is further configured to provide the deducted number of healthcare credits to the healthcare provider.

11. A computer implemented method for determining and generating a patient healthcare plan, the method comprising the steps of:
receiving input from a user through a client device, wherein the input comprises data for populating a patient data record;
identifying from a database, via a computing device processor, an impact factor corresponding to the data populating the patient data record;
calculating a healthcare cost for a time period based on the identified impact factor; and
generating a patient healthcare plan having a healthcare item and having a number of healthcare credits that are accrued over the time period.

12. The method of claim 11, wherein the impact factor is selected from the group consisting of a geo-location factor, a retail cost factor, and a price index factor.

13. The method of claim 11, wherein the healthcare credits are exchangeable for the healthcare item.

14. The method of claim 11, wherein two or more impact factors corresponding to the data populating the patient data record are identified from the database, and wherein each impact factor is additive to the calculated healthcare cost for a time period.

15. The method of claim 11, wherein two or more patient healthcare plans are generated, and wherein each healthcare plan has a different number of healthcare credits that are accrued over the time period.

16. The method of claim 15, further comprising the steps of: receiving input from a user through a client device, wherein the input identifies a healthcare plan selected by the user; and generating a contract for the selected healthcare plan.

17. The method of claim 11, wherein the time period is selected from the group consisting of: a week, two weeks, three weeks, four weeks, a month, two months, three months, four months, five months, six months, and a year.

18. The method of claim 11, wherein the healthcare cost for a time period is calculated by retrieving a base cost per time period, the base cost per time period associated with the data populating the patient data record, and wherein the base cost per time period and identified impact factor are multiplied together to calculate the healthcare cost for a time period.

19. The method of claim 11, further comprising the steps of: receiving a service request for the healthcare item; and deducting a number of healthcare credits associated with the healthcare item from the accrued healthcare credits.

20. The method of claim 19, wherein the service request for the healthcare item is received from a healthcare provider, and wherein the method further comprises the step of providing the deducted number of healthcare credits to the healthcare provider.1
